(19) United States
(12) Patent Application Publication
(10) Pub. No.: US 2006/0079907 A1
(43) Pub. Date: Apr. 13, 2006

(54) Guide device for a surgical machining tool

(75) Inventors: Roland Boettiger, Rietheim-Weilheim (DE); Gerd Ruthe, Tuttlingen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/150,867

(22) Filed: Jun. 9, 2005

Related U.S. Application Data
(63) Continuation of application No. PCT/EP03/13747, filed on Dec. 5, 2003.

(30) Foreign Application Priority Data
Dec. 13, 2002 (DE) 102 58 322.6

(51) Int. Cl. A61F 5/00

(52) U.S. Cl. 606/87

(57) ABSTRACT

In a guiding device for a surgical machining tool, which, in particular, is securable to a bone to be worked on and comprises a guiding element which guides the machining tool and is mounted on the guiding device so as to be displaceable in one plane, in order to improve the visibility of the operating site, it is proposed that the guiding element be connected to a frame of the guiding device by at least one lever by means of a pivot whose axis of rotation extends perpendicularly to the guiding plane.

The invention relates to a guiding device for a surgical machining tool, which, in particular, is securable to a bone to be worked on, with a guiding element which guides the machining tool and is mounted on the guiding device so as to be displaceable in one plane only.

Such a guiding device is known, for example, from DE 202 02 615 U1. With this guiding device it is possible to guide a machining tool, for example, a high-speed end milling cutter, by means of a guide sleeve in such a way in a plane that bone material can thereby be removed along a plane. A machining of the bone may also be carried out in other planes by changing parts of the guiding device, so that, for example, the distal end of a femur may be prepared for receiving a corresponding femur endoprosthesis. In the known device, the guiding element is guided in a slot-shaped guide of the guiding device and can be pushed back and forth and pivoted in this slot-shaped guide by the user. In individual cases, the slot-shaped guide may cause the operator's view of the operating site to be impaired.

The object of the invention is, therefore, to so design a generic guiding device that, on the one hand, reliable guidance of the machining tool is ensured and, on the other hand, optimum visibility of the operating site is maintained.

This object is accomplished, in accordance with the invention, in a guiding device of the kind described at the outset in that the guiding element is connected to a frame of the guiding device by at least one lever by means of a pivot whose axis of rotation extends perpendicularly to the guiding plane.

The use of such a rotatable lever likewise guides the guiding element in the guiding plane, but the lever can be arranged in such a way outside of the guiding plane that impairment of the operator's field of vision is avoided.

Whereas it is, in principle, possible to pivotably connect the guiding element to the frame in this way with only one lever, it is provided, in accordance with a particularly preferred embodiment, that two levers are mounted on the frame of the guiding device so as to be rotatable about axes of rotation extending parallel to each other, and the two levers are, in turn, connected to the guiding element so as to be rotatable about axes of rotation extending parallel to these axes of rotation. The guiding element is thus mounted by means of two levers or swivel levers in a very reliable, substantially play-free manner, so that it is ensured that the machining tool will be guided exactly in the guiding plane even under tough conditions.

The lines connecting the two pivots of the two levers may extend parallel to each other. This then results in a parallelogram guidance in which the guiding element is displaced parallel to itself on a circular path whose radius is defined by the spacing of the axes of rotation of the levers. In another embodiment, however, it can be provided that the lines connecting the two pivots of the two levers extend at an incline to each other. Upon pivoting the levers, this results in a simultaneous pivoting of the guiding element, with this pivoting movement depending on how large the angle of the two levers is in relation to each other.

It is advantageous for the guiding element to comprise two legs extending at an angle to each other in the guiding plane. In particular, the angle between the two legs may be between 75° and 105°, preferably about 90°. By means of this arrangement it is possible to allow the guiding element to project from the connection line of the mounting of the guiding element on the levers. In this way, one can use relatively long levers and nevertheless move the guidance of the machining tool in the direction towards the machining location.

The guiding element itself may guide the machining tool in any way. It is particularly advantageous for the guiding element to carry a guide sleeve for receiving the machining tool.

This guide sleeve may be fixedly connected to the guiding element, but it is also possible for the guide sleeve to be mounted on the guiding element so as to be rotatable about an axis of rotation which extends parallel to the axis of rotation of the lever or levers. In this way, the user is free to pivot the guide sleeve and thus the machining tool in any direction in the guiding plane.

If, in accordance with a preferred embodiment the longitudinal axis of the guide sleeve lies in the guiding plane, a surface which extends parallel to the guiding plane can be machined with the machining tool. In principle, it is also possible to incline the longitudinal axis of the guide sleeve in relation to the guiding plane, so that the machined surface is then also at an incline to the guiding plane.

In the case of an angled guiding element, the guide sleeve may preferably be held at the junction of the two legs on the guiding element, so that the guide sleeve can be positioned as closely as possible to the machining location.

It is advantageous for the pivotability of the lever or levers to be limited by a stop, which is preferably adjustable in its position.

The lever or levers or also the guiding element may comprise openings, in particular, in the form of elongate slots. On the one hand, their weight is thereby reduced. On the other hand, these openings provide fields of vision for the operator, through which he can observe the operating site.

In a simple configuration of the guiding device the guiding element is movable by the lever or levers in one guiding plane. If machining of the bone is desired in different planes, in particular, in differently orientated planes, as is the case, for example, when preparing a distal femur, it is then advantageous for the lever or levers to be mountable on the frame so as to be pivotable about various axes of rotation. There are various possibilities for realizing this, for example, the position of the pivots of the lever or levers on the frame may be adjustable. The pivots may be mounted on a part of the frame which is, for example, pivotable in relation to the entire guiding device.

In another configuration it is provided that several pivot locations with different axes of rotation, at which the lever or levers are selectively mountable, are arranged on the frame.

In a further possible configuration the lever or levers is or are insertable with their pivots in various receivers of the frame.

It may also be provided that those parts of the pivots of the lever or levers that are arranged on the frame are insertable in a different position into the same receivers on the frame.

In all cases it is possible for the levers to be mounted in different positions and with different orientations on the same frame and thus on the same guiding device, so that also different guiding planes are obtained, which are adapted to the desired machining surfaces. It is thus possible to also carry out complicated machining procedures on a bone with only one guiding device on which the guiding element is mounted by means of the levers so as to be pivotable in different planes.

The following description of preferred embodiments of the invention serves in conjunction with the drawings for further explanation. The drawings show:

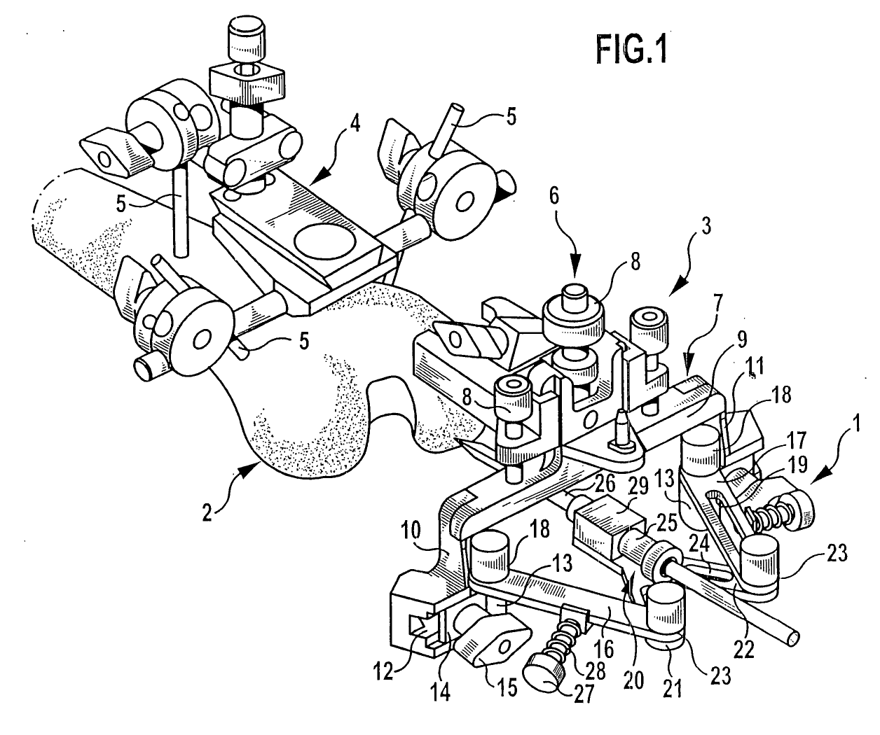

FIG. 1: a perspective view of a modular guiding device held on the distal femur for an end milling cutter-type machining tool prior to assembly of the modules of the guiding device;

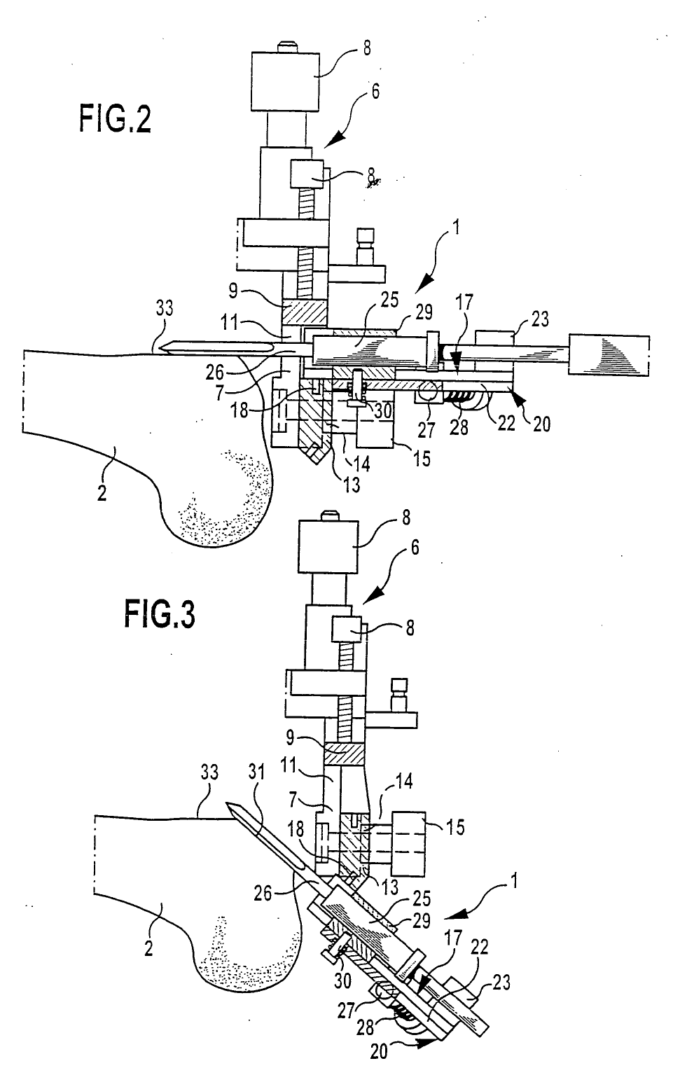

FIG. 2: a cross-sectional view of the frame part of the guiding device of FIG. 1 with the guiding element in a first position;

FIG. 3: a view similar to FIG. 2 with the guiding element in a second position;

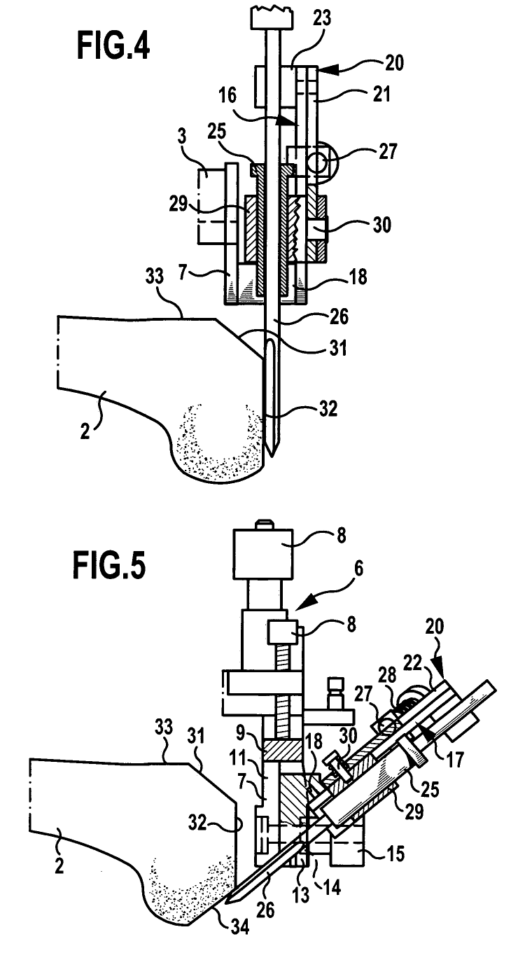

FIG. 4: a schematic view similar to FIG. 3 with the guiding element in a third position;

FIG. 5: a view similar to FIG. 3 with the guiding element in a fourth position;

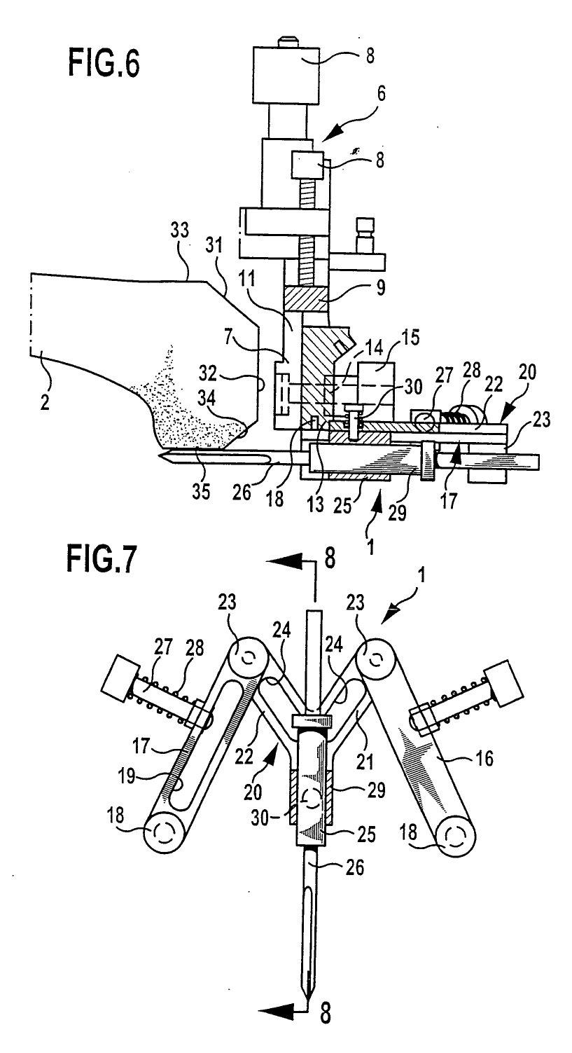

FIG. 6: a view similar to FIG. 2 with the guiding element in a fifth position;

FIG. 7: a plan view of the levers, the guiding element and the machining tool guided therein in a middle position;

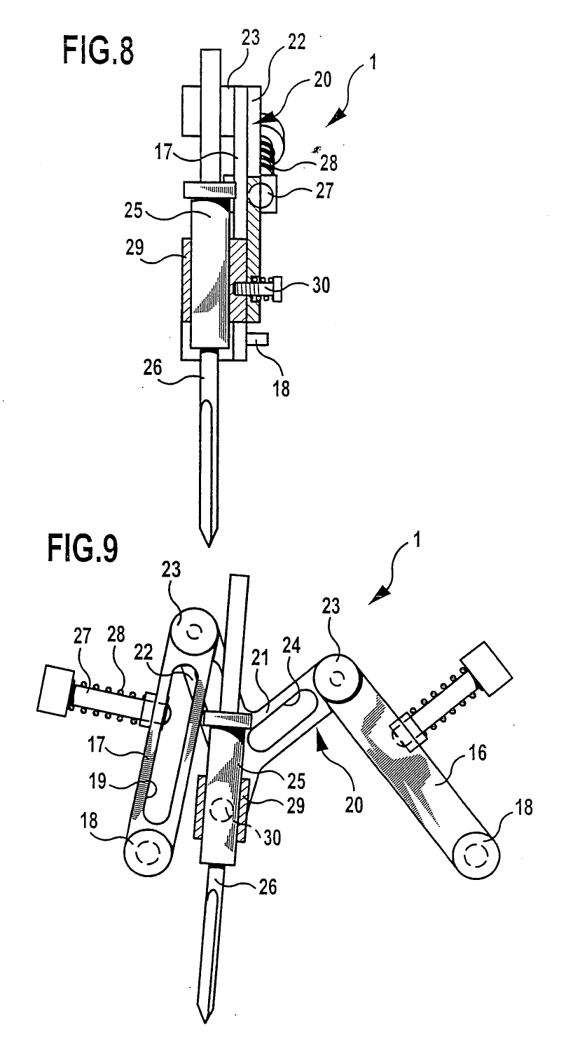

FIG. 8: a sectional view along line 8-8 in FIG. 7;

FIG. 9: a view similar to FIG. 7 with the guiding element in a position in which it is pivoted out of the middle position;

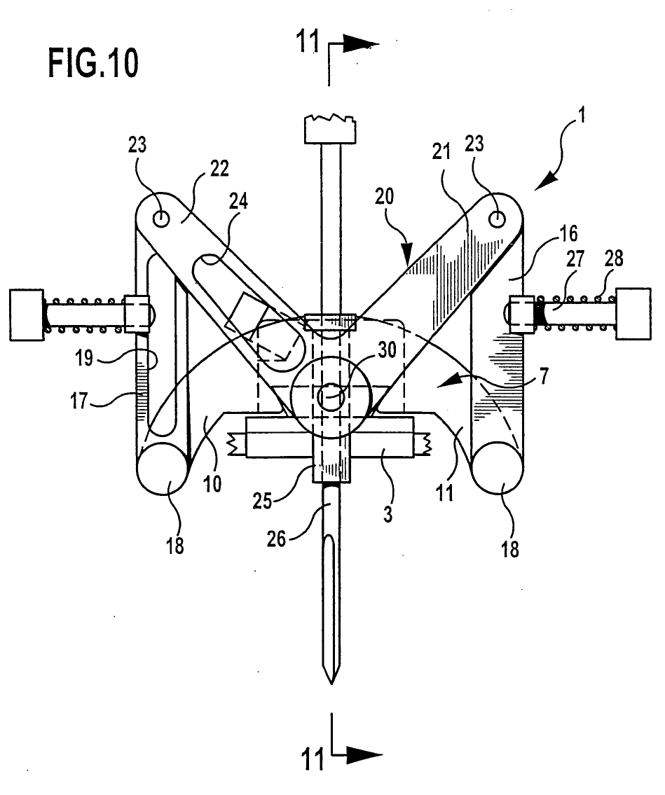

FIG. 10: a view similar to FIG. 7 in another preferred embodiment of a guiding device;

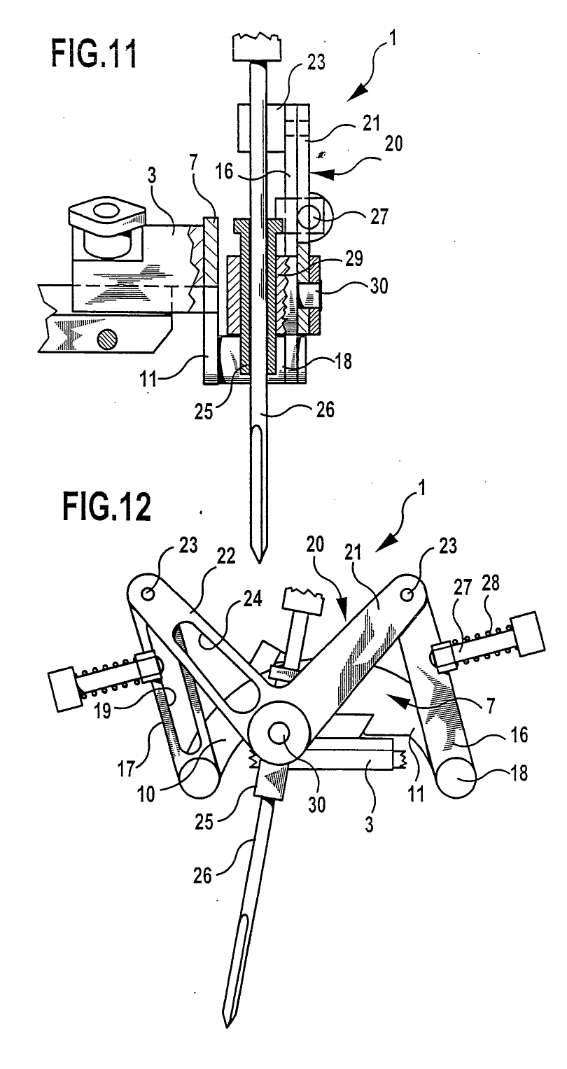

FIG. 11: a sectional view along line 11-11 in FIG. 10; and

FIG. 12: a view similar to FIG. 10 with a guiding element pivoted out of the middle position.

The invention will be explained with reference to a device used for preparing the distal femur. However, it goes without saying that the described device may also be used for working on other bones of the body where it is important that the bones be machined along a plane, i.e., when making saw cuts or when machining a surface using a high-speed end milling cutter.

Very different devices may be used for securing the new guiding device 1 to the distal femur 2. In the present embodiment, the guiding device 1 is secured to the distal femur 2 by means of a holding device 3 of modular construction, which comprises a platform 4 which is held by means of post-like pins 5 on the femur 2. There can be pushed onto this platform 4 an adjusting module 6 on which a substantially U-shaped holding bracket 7 is arranged. The holding bracket 7 may be aligned in the desired manner relative to the femur 2 by means of different setting members 8 which are not explained in further detail herein. Insofar the holding device 3 corresponds to the holding device described, for example, in DE 202 02 615 U1, to which reference is expressly made herein.

The holding bracket 7 comprises a horizontal bridge 9 and two legs 10, 11 projecting perpendicularly therefrom. Arranged at the free ends of the legs 10, 11 and extending parallel to the bridge 9 are groove-shaped receivers 12 into which bearing blocks 13 are insertable in a guided manner from the side. These bearing blocks 13 may be fixed in the receivers 12 with clamping plates 14. The clamping plates 14 are tightened by clamping screws 15.

The described holding device 3 with the holding bracket 7 forms a frame on which the actual guiding device 1 is mounted, namely by means of the bearing blocks 13.

This guiding device 1 comprises two rod-shaped levers 16, 17, which are respectively mounted for rotation at one end on one bearing block 13 respectively. The axis of rotation of the corresponding pivot 18 extends parallel to the legs 10, 11 and lies close to these legs 10, 11, as the bearing blocks 13 protrude only a little from the groove-shaped receivers 12.

Both levers 16, 17 are of straight-lined and plate-shaped design. In one of the two levers there is an opening 19 in the form of an elongate slot. In principle, both levers could be designed with or without an opening.

At the end remote from the pivot 18, both levers 16, 17 are pivotably connected to a guiding element 20 designed as a two-armed lever with two lever arms 21, 22, which include an angle of approximately 90° with each other. The pivots 23 thus formed have axes of rotation which extend parallel to the axes of rotation of the pivots 18. The pivots 23 are positioned at the free ends of the levers 16, 17 or the lever arms 21, 22. The length of the lever arms 21, 22 is so selected that the spacing of the pivots 23 from each other is smaller than the spacing of the pivots 18 from each other. The levers 16, 17 thus extend at an incline to each other (FIG. 7). Elongate slot-like openings 24 are likewise provided in the lever arms 21, 22.

A guide sleeve 25 is mounted at the junction of the two lever arms 21, 22 on one side of the plate-shaped guiding element 20. This mounting may be a fixed mounting, but it is also possible to slide the guide sleeve 25 into a receiving aperture and secure it there releasably by frictional forces. The guide sleeve 25 serves to receive the shaft of a high-speed end milling cutter 26. The guide sleeve 25 may be specially designed for this, for example, by way of ball bearings resting against the shaft of the end milling cutter 26. This is not explained in further detail herein.

In a first embodiment the guide sleeve 25 is rigidly connected to the guiding element 20. The longitudinal axis of the guide sleeve 25 then extends on the bisector of the angle of the two lever arms 21, 22.

With the described guiding device 1, the operator can push the end milling cutter 26 into the guide sleeve 25 and then by pivoting the end milling cutter 26 in a plane extending parallel to the guide element 20 pivot the guide sleeve 25 in a guided manner in this plane, which in the following is called guiding plane. The guiding is effected by the two levers 16, 17 which, owing to their inclination to each other, pivot the guiding element 20 as well while pivoting. This pivoting of the guiding element 20 depends on the length ratios and on the inclined position of the two lever arms 21, 22 in relation to each other. The guiding device 1 is shown in a middle position in FIG. 7, in a pivoted position in FIG. 9. By means of this guided pivoting, the end milling cutter 26 covers in a defined manner a certain area in the guiding plane and can work on this area, for example, at the dorsal side of the distal femur, as shown in FIG. 2. In this case, the pivoting range is determined by the guide sleeve 25 and may be limited by the pivoting movement of the lever arms 21, 22 being limited. For this purpose, the levers 16, 17 carry adjustable stop pins 27, which are held on the levers 16, 17 transversely to the longitudinal direction of the levers 16, 17 and can be pushed in to a greater or lesser depth, so that they strike the guiding element 20 and thereby limit the movability of the guide sleeve 25 and thus the area covered by the end milling cutter 26 when the levers 16, 17 are pivoted. In the drawings the stop pins 27 are all shown in an inoperative, pulled-out position. They may, however, be pushed in against the action of helical springs 28 surrounding them.

Whereas in the embodiment of FIGS. 1 to 9 the levers 16, 17 extend at an incline to each other, in the embodiment of FIGS. 10 to 12 a configuration of the levers 16, 17 is chosen wherein these are always arranged parallel to each other, i.e., the spacing of the pivots 23 from each other is exactly the same as the spacing of the pivots 18 from each other. The guiding element 20 is thus always moved parallel to itself when the levers 16, 17 are pivoted.

The guide sleeve 25 may be mounted on the guiding element 20 so as to be pivotable about an axis of rotation which extends parallel to the axes of rotation of the pivots 18 and 23, so that the guide sleeve 25 may not only be displaced together with the guiding element 20 in the guiding plane, but the guide sleeve 25 may also be rotated about this axis of rotation relative to the guiding element 20 in the guiding plane. For this purpose, a bearing block 29 receiving the guide sleeve 25 is rotatably mounted by means of a bearing pin 30 in the junction area of the lever arms 21 and 22.

With the arrangement shown in FIGS. 1 and 2 a certain guiding plane is obtained, i.e., with this arrangement a single surface is machinable on the femur 2, in the case of FIGS. 1 and 2 on the distal femur, with the guiding device 1.

To enable machining of other surfaces, provision is made for the guiding device 1 to be securable in different positions on the holding device 3.

This is, for example, achievable by different bearing possibilities being provided for the levers 16, 17 on the bearing blocks 13, for example, in accordance with the illustration in FIG. 3 at the top end of the bearing block 13 and with an inclined axis of rotation at the bottom end of the bearing block 13. It is thus possible to fix the guiding device 1 on the guiding device at an angle corresponding to the inclination of this axis of rotation and to thereby produce an inclined guiding plane by means of which an inclined surface 31 may be machined on the femur.

Different bearing blocks 13 may be used, so that further orientations are possible. For example, in FIG. 4 an orientation is shown, with which a distal femur surface 32 may be machined, which extends perpendicularly to the dorsal surface 33 which can be reached with the arrangement of FIGS. 1 and 2. With the configuration according to FIG. 5, an inclined anterior surface 34 may be machined, with the arrangement according to FIG. 6 an anterior surface 36 extending perpendicularly to the distal surface 32.

It is possible to join the guiding device 1 permanently to the associated bearing blocks 13 and to exchange these together with a guiding device 1 when other orientations are desired. It is, however, also possible to join the guiding device 1 to other bearing blocks 13 or to use other bearing points on these bearing blocks 13 in each case so as to obtain the various orientations with a single guiding device 1 and different bearing blocks 13.

In another embodiment, not shown in the drawings, it is also possible for the guiding devices 1 to be of an adjustable design. For example, the holding brackets 7 could be of pivotable design, and different setting possibilities are conceivable here.

The mounting of the guide sleeve 25 on the frame using the levers 16, 17 results in a very safe guidance of the guide sleeve 25, which is possible in a substantially play-free manner and makes a reliable guiding of the guide sleeve in the guiding plane and along a precisely defined path of displacement possible for the operator. This increases the safety during the machining procedure and owing to the special construction also makes it possible for the operator to observe the operating site during the entire machining procedure.

1-20. (canceled)

21. Guiding device for a surgical machining tool with a guiding element which guides the machining tool and is mounted on the guiding device so as to be displaceable in one plane only, wherein two levers are mounted on a frame of the guiding device by means of a pivot so as to be rotatable about axes of rotation extending parallel to each other and perpendicularly to the guiding plane, and the said levers are, in turn, connected to the guiding element so as to be rotatable about axes of rotation extending parallel to these axes of rotation.

22. Guiding device in accordance with claim 21, wherein the lines connecting the two pivots of the two levers extend parallel to each other.

23. Guiding device in accordance with claim 21, wherein the lines connecting the two pivots of the two levers extend at an incline to each other.

24. Guiding device in accordance with claim 21, wherein the guiding element comprises legs extending at an angle to each other in the guiding plane.

25. Guiding device in accordance with claim 24, wherein the angle between the two legs lies between 75° and 105°.

26. Guiding device in accordance with claim 21, wherein the guiding element carries a guide sleeve which receives the machining tool.

27. Guiding device in accordance with claim 24, wherein the guiding element carries a guide sleeve which receives the machining tool.

28. Guiding device in accordance with claim 26, wherein the guide sleeve is fixedly connected to the guiding element.

29. Guiding device in accordance with claim 26, wherein the guide sleeve is mounted on the guiding element so as to be rotatable about an axis of rotation which extends parallel to the axis of rotation of the lever or levers.

30. Guiding device in accordance with claim 26, wherein the longitudinal axis of the guide sleeve lies in the guiding plane.

31. Guiding device in accordance with claim 26, wherein the guide sleeve is held at the junction of the two legs on the guiding element.

32. Guiding device in accordance with claim 21, wherein the pivotability of the lever or levers is limited by a stop.

33. Guiding device in accordance with claim 32, wherein the stop is adjustable in its position.

34. Guiding device in accordance with claim 21, wherein the lever or levers and/or the guiding element have openings.

35. Guiding device in accordance with claim 34, wherein the openings are formed as elongate slots.

36. Guiding device in accordance with claim 21, wherein the lever or levers are mountable on the frame so as to be pivotable about various axes of rotation.

37. Guiding device in accordance with claim 36, wherein the position of the pivots of the lever or levers on the frame is adjustable.

38. Guiding device in accordance with claim 36, wherein several pivot locations with different axes of rotation are arranged on the frame for optional mounting of the lever or levers thereat.

39. Guiding device in accordance with claim 36, wherein the lever or levers is or are insertable with its or their pivot in various receivers of the frame.

40. Guiding device in accordance with claim 36, wherein those parts of the pivot of the lever or levers that are arranged on the frame are insertable in a different position into the same receiver on the frame.
